(19) United States
(12) Patent Application Publication
(10) Pub. No.: US 2026/0250776 A1
(43) Pub. Date: Aug. 27, 2026

(54) BCOR AND BRD4 GENE FUSIONS AND USES THEREOF

(71) Applicant: Foundation Medicine, Inc., Boston, MA (US)

(72) Inventor: Douglas I. LIN, Needham, MA (US)

(73) Assignee: Foundation Medicine, Inc., Boston, MA (US)

(21) Appl. No.: 19/163,121

(22) PCT Filed: Mar. 7, 2024
(86) PCT No.: PCT/US2024/018905

§ 371 Date: Sep. 8, 2025

Related U.S. Application Data
(60) Provisional application No. 63/451,439, filed on Mar. 10, 2023.

(51) Int. Cl. C12Q 1/6886

(52) U.S. Cl. CPC C12Q 1/6886; C12Q 2600/156

(57) ABSTRACT

The present disclosure relates generally to biomarkers of embryonic-type neuroectodermal tumors (ENETs, formerly PNETs), as well as methods of diagnosis and treatment related thereto.

### CROSS-REFERENCE TO RELATED APPLICATION

This application claims the benefit of U.S. Provisional Application No. 63/451,439, filed Mar. 10, 2023, which is hereby incorporated by reference in its entirety.

### FIELD

The present disclosure relates generally to biomarkers of embryonic-type neuroectodermal tumors (ENETs, formerly PNETs), as well as methods of diagnosis and treatment related thereto.

### BACKGROUND

Increasing evidence supports the important diagnostic value and therapeutic roles of recurrent chromosomal translocations in epithelial solid tumors. Testicular ENET arising from germ cell tumors (GCT) is a relatively common type of somatic transformation in GCT, characterized by the overgrowth of immature neuroectodermal components. Due to the high prevalence of resistance to chemotherapy, ENET is usually associated with poor prognosis and limited therapeutic options, particularly when patients develop disease recurrence or metastasis.

Although it is well accepted that testicular ENET does not harbor ESWR1-FLI1 rearrangements typical of Ewing sarcoma, knowledge of key events driving this somatic transformation is limited to the paucity of comprehensive genomic data in GCT. The present disclosure describes a characterization of genomic features of these rare tumors to aid in molecular classification and treatment.

### SUMMARY OF THE INVENTION

In certain aspects, provided herein are methods for assessing or diagnosing a cancer in an individual, comprising detecting a BRD4-MAU2 fusion and/or a BCOR-CLIP2 fusion in a sample from an individual having a cancer, wherein detecting the BRD4-MAU2 fusion and/or the BCOR-CLIP2 fusion in the sample indicates that the cancer is likely an embryonic-type neuroectodermal tumor (ENET).

In other aspects, provided herein are methods for treating a cancer, comprising: (a) detecting a BRD4-MAU2 fusion in a sample from an individual having a cancer, and (b) administering a BET bromodomain inhibitor to the individual based, at least in part, on detecting the BRD4-MAU2 fusion in the sample.

In some embodiments, the BRD4-MAU2 fusion: (a) comprises a fusion of a BRD4 gene, or a portion thereof, fused to a MAU2 gene, or a portion thereof; (b) comprises or results from a BRD4 breakpoint in BRD4 exon 3; (c) comprises a fusion of BRD4 exon 3, or a portion thereof, fused to an exon of MAU2, or a portion thereof; (d) results from a breakpoint within chromosomal coordinates chr19:15379580-15379772 and/or chr19:19434185-19434416; and/or (e) is a BRD4-MAU2 fusion as described in Table 3.

In some embodiments, the BCOR-CLIP2 fusion: (a) comprises, in the 5′ to 3′ direction, a fusion of a BCOR gene, or a portion thereof, fused to a CLIP2 gene, or a portion thereof; (b) comprises or results from a BCOR breakpoint in BCOR exon 9 and/or a CLIP2 breakpoint in CLIP2 intron 2; (c) comprises, in the 5′ to 3′ direction, a fusion of BCOR exon 9, or a portion thereof, fused to CLIP2 exon 3, or a portion thereof; (d) comprises, in the 5′ to 3′ direction, exons 1-8 and exon 9, or a portion thereof, of BCOR fused to exon 3, or a portion thereof, and exons 4-17 of CLIP2; (e) results from a breakpoint within chromosomal coordinates chrX:39922132-39922319 and/or chr7:73738770-73738988; and/or (f) is a BCOR-CLIP2 fusion as described in Table 3.

In some embodiments, detecting the BRD4-MAU2 fusion in the sample identifies the individual or the cancer as likely to respond to a BET bromodomain inhibitor treatment.

In some embodiments, the methods comprise administering the BET bromodomain inhibitor to the individual based, at least in part, on detecting the BRD4-MAU2 fusion in the sample.

Exemplary BET bromodomain inhibitors include, without limitation, JQ1 (for example, CAS Registry Number: 1268524-70-4), I-BET762 (for example, CAS Registry Number: 1260907-17-2), OTX015 (for example, CAS Registry Number: 202590-98-5), I-BET151 (for example, CAS Registry Number: 1300031-49-5), RVX-208 (for example, CAS Registry Number: 1044870-39-4), MS417 (for example, CAS Registry Number: 916489-36-6), ABBV-075 (for example, CAS Registry Number: 1445993-26-9), ABBV-744 (for example, CAS Registry Number: 2138861-99-9), SJ432 (for example, CAS Registry Number: 2230677-55-9), AZD5153 (for example, CAS Registry Number: 1869912-40-2), and INCB054329 (for example, CAS Registry Number: 1628607-64-6).

In some embodiments, the BET bromodomain inhibitor is JQ1, I-BET762, OTX015, I-BET151, RVX-208, MS417, ABBV-075, ABBV-744, SJ432, AZD5153, INCB054329, or any combination thereof.

In some embodiments, the BCOR-CLIP2 fusion results in a loss of function of BCOR. In some embodiments, the methods comprise administering an anti-cancer therapy to the individual based, at least in part, on detecting the BCOR-CLIP2 fusion in the sample.

In some embodiments, the cancer is a testicular tumor, optionally a testicular tumor arising from a germ cell tumor. In some embodiments, the cancer is a testicular embryonic-type neuroectodermal tumor arising from a germ cell tumor. In some embodiments, the cancer is advanced and/or metastatic.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

FIG. 1 shows genetic alterations that were identified in the 12 cases of ENETs arising from testicular GCTs.

FIGS. 2A-2D show representative images of liver metastases from an ENET case (case number 12) arising from testicular teratoma. FIG. 2A shows that the tumor cells were predominantly uniform, oval to spindle-shaped with scant cytoplasm and delicate chromatin.

FIG. 2B shows perivascular pseudorosettes were easily identified. FIG. 2C shows areas with nuclear pleomorphism and moderate pale eosinophilic cytoplasm. FIG. 2D shows focal myxoid or fibrillary formation.

FIG. 3 provides chromosomal copy number changes observed in the 12 patients with advanced testicular ENETs of GCT origin in this study.

The following exemplary embodiments are representative of some aspects of the invention:
Embodiment 1. A method for assessing or diagnosing a cancer in an individual, comprising detecting a BRD4-MAU2 fusion and/or a BCOR-CLIP2 fusion in a sample from an individual having a cancer, wherein detecting the BRD4-MAU2 fusion and/or the BCOR-CLIP2 fusion in the sample indicates that the cancer is likely an embryonic-type neuroectodermal tumor (ENET).
Embodiment 2. The method of embodiment 1, wherein the BRD4-MAU2 fusion:
   (a) comprises a fusion of a BRD4 gene, or a portion thereof, fused to a MAU2 gene, or a portion thereof;
   (b) comprises or results from a BRD4 breakpoint in BRD4 exon 3;
   (c) comprises a fusion of BRD4 exon 3, or a portion thereof, fused to an exon of MAU2, or a portion thereof;
   (d) results from a breakpoint within chromosomal coordinates chr19:15379580-15379772 and/or chr19:19434185-19434416; and/or
   (e) is a BRD4-MAU2 fusion as described in Table 3.
Embodiment 3. The method of embodiment 1, wherein the BCOR-CLIP2 fusion:
   (a) comprises, in the 5′ to 3′ direction, a fusion of a BCOR gene, or a portion thereof, fused to a CLIP2 gene, or a portion thereof;
   (b) comprises or results from a BCOR breakpoint in BCOR exon 9 and/or a CLIP2 breakpoint in CLIP2 intron 2;
   (c) comprises, in the 5′ to 3′ direction, a fusion of BCOR exon 9, or a portion thereof, fused to CLIP2 exon 3, or a portion thereof;
   (d) comprises, in the 5′ to 3′ direction, exons 1-8 and exon 9, or a portion thereof, of BCOR fused to exon 3, or a portion thereof, and exons 4-17 of CLIP2;
   (e) results from a breakpoint within chromosomal coordinates chrX:39922132-39922319 and/or chr7:73738770-73738988; and/or
   (f) is a BCOR-CLIP2 fusion as described in Table 3.
Embodiment 4. The method of embodiment 1 or embodiment 2, wherein detecting the BRD4-MAU2 fusion in the sample identifies the individual or the cancer as likely to respond to a BET bromodomain inhibitor treatment.
Embodiment 5. The method of embodiment 4, wherein the BET bromodomain inhibitor is JQ1, I-BET762, OTX015, I-BET151, RVX-208, MS417, ABBV-075, ABBV-744, SJ432, AZD5153, INCB054329, or any combination thereof.
Embodiment 6. The method of embodiment 4 or embodiment 5, further comprising administering the BET bromodomain inhibitor to the individual based, at least in part, on detecting the BRD4-MAU2 fusion in the sample.
Embodiment 7. The method of embodiment 1 or embodiment 3, wherein the BCOR-CLIP2 fusion results in a loss of function of BCOR.
Embodiment 8. The method of any one of embodiments 1, 3 and 7, further comprising administering an anti-cancer therapy to the individual based, at least in part, on detecting the BCOR-CLIP2 fusion in the sample.
Embodiment 9. The method of any one of embodiments 1-8, wherein the cancer is a testicular tumor, optionally a testicular tumor arising from a germ cell tumor.
Embodiment 10. The method of any one of embodiments 1-9, wherein the cancer is a testicular embryonic-type neuroectodermal tumor arising from a germ cell tumor.
Embodiment 11. The method of any one of embodiments 1-10, wherein the cancer is advanced and/or metastatic.
Embodiment 12. A method for treating a cancer, comprising:
   (a) detecting a BRD4-MAU2 fusion in a sample from an individual having a cancer, and
   (b) administering a BET bromodomain inhibitor to the individual based, at least in part, on detecting the BRD4-MAU2 fusion in the sample.

### Experimental Methods

A retrospective database search of a Clinical Laboratory Improvement Amendments (CLIA)- and College of American Pathologists (CAP)-certified reference molecular laboratory was performed for ENETs arising from testicular GCTs that had previously undergone next-generation sequencing (NGS)-based comprehensive genomic profiling (CGP) during the course of clinical care. Clinicopathological and genomic data was centrally re-reviewed.

Twelve cases of ENETs arising from testicular GCTs that had previously undergone NGS-based CGP during the course of clinical care were identified. The CGP involved targeted hybrid capture DNA- and RNA-based CGP.

### Results

The molecular features of 12 advanced testicular ENETs of GCT origin were characterized.

Patient age ranged from 24 to 49 years, with a mean and median of 36 and 38 years, respectively. CGP was performed in 1 primary tumor and 11 metastases, whose submitting diagnoses were testicular ENET with or without an associated classical GCT component.

Table 1 provides a summary of the characteristics of the 12 patients with advanced testicular ENETs of GCT origin in this study.

TABLE 1
Characteristics of patients with advanced testicular ENETs of GCT origin.

| Case | Age | Diagnosis |
|---|---|---|
| 1 | 25 | Metastatic ENET with atypical glial differentiation and teratoma |
| 2 | 38 | Metastatic mixed germ cell tumor with ENET |
| 3 | 42 | Recurrent ENET previously transformed from teratoma |
| 4 | 48 | Metastatic germ cell tumor composed predominantly of teratoma and a small |
| | | focus of ENET |
| 5 | 37 | Metastatic germ cell tumor composed predominantly of ENET and small |
| | | foci of teratoma and YST |
| 6 | 26 | Metastatic ENET consistent with component of known germ cell tumor. |
| 7 | 42 | ENET morphologically similar to prior component of mixed germ cell tumor |
| 8 | 32 | Metastatic ENET and teratoma |
| 9 | 39 | Metastatic malignancy with neuroectodermal differentiation |
| | | morphologically similar to prior tumor arising from teratoma |
| 10 | 49 | Predominantly ENET (95%) and teratoma (5%) |
| 11 | 24 | Metastatic ENET, central type (embryonic-type neuroectodermal tumor) |
| 12 | 31 | Metastatic ENET with known history of ENET arising from teratoma |

Table 2 shows the clinicopathological features of the 12 cases of ENETs arising from testicular GCTs in this study.

TABLE 2
Clinicopathological features of the study cases.

| Median age | 38 years (ranging from 24 to 49 years) | | |
|---|---|---|---|
| Primary | Teratoma (n = 7) | Mixed GCT (n = 3) | Uncertain (n = 2) |
| GCT | | | |

| | | | | | |
|---|---|---|---|---|---|
| Sequenced | Testis | Lymph | Lung | Liver | Other* |
| tumor site | (n = 1) | nodes | (n = 2) | (n = 2) | (n = 4) |
| | | (n = 3) | | | |

*Including omentum (n = 1), paraaortic (n = 1), paraspinal (n = 1) and retroperitoneum (n = 1).

All tumors harbored gain of chromosome 12p, often with KRAS, CCND2 and KMD5A co-amplification, supporting a germ cell origin, and all were negative for EWSR1 fusions. The tumors were microsatellite stable and exhibited low tumor mutational burden.

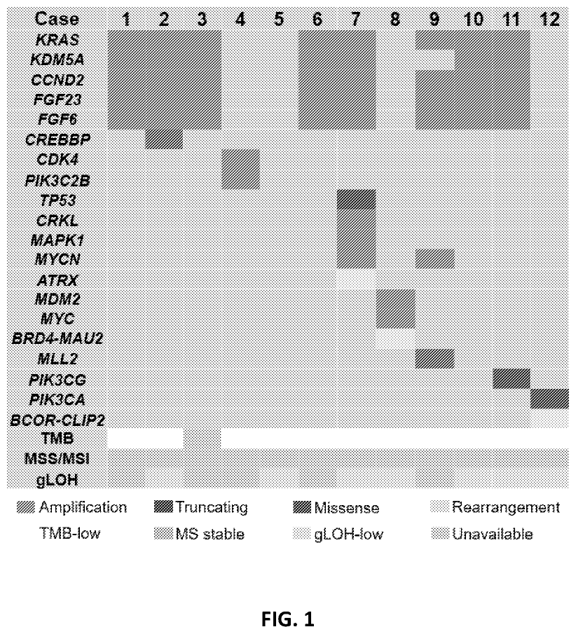

FIG. 1 shows genetic alterations that were identified in the 12 cases of ENETs arising from testicular GCTs. As shown in FIG. 1, testicular ENETs arising from GCTs were molecularly heterogeneous.

A subset of testicular ENETs could be stratified by genetic alterations, including MYCN/MYC amplification with concurrent suppression of the p53 pathway, and activation of the PI3K pathway. 3 tumors (25%) exhibited MYCN or MYC amplification with co-occurring inactivation of the p53 pathway via either TP53 mutations or MDM2 amplification in 2 tumors. 3 additional tumors (25%) had activation of the PI3K pathway via PIK3CA and PIK3CG mutations or PIK3C2B amplification; one tumor with co-occurring CDK4 amplification.

Fusions or rearrangements were detected in 3 tumors (25%), including an internal truncating ATRX rearrangement with no partner, as well as novel BRD4-MAU2 and BCOR-CLIP2 fusions. Table 3 provides certain characteristics of the BRD4-MAU2 and BCOR-CLIP2 fusions.

TABLE 3
Characteristics of BRD4-MAU2 and BCOR-CLIP2 fusions.

| Fusion | Description |
|---|---|
| BRD4-MAU2 | BRD4(NM_014299) rearrangement exon 3, chr19 deletion |
| fusion | fragment: BRD4 (NM_014299): 5′ rearrangement breakpoint exon |
| | 3. Position 1: chr19: 15379580-15379772. Position 2: |
| | chr19: 19434185-19434416. |
| BCOR-CLIP2 | BCOR(NM_017745)-CLIP2(NM_003388) fusion (B9*; C3). 5′- |
| fusion | BCOR(ex1-9 NM_017745)-CLIP2(ex3-17 NM_003388) |
| | Breakpoints BCOR exon 9, CLIP2 intron 2; Reciprocal: no. Position |
| | 1: chrX: 39922132-39922319. Position 2: chr7: 73738770-73738988. |

The 2 cases with BRD4 and BCOR fusions were reminiscent of the emerging central nervous system (CNS) embryonal tumor (CNS-PNET) entities that are defined by molecular alterations, such as CNS HGNET-BCOR. The results described herein indicate a common mechanism underlying the pathogenesis of ENETs from different anatomic sites.

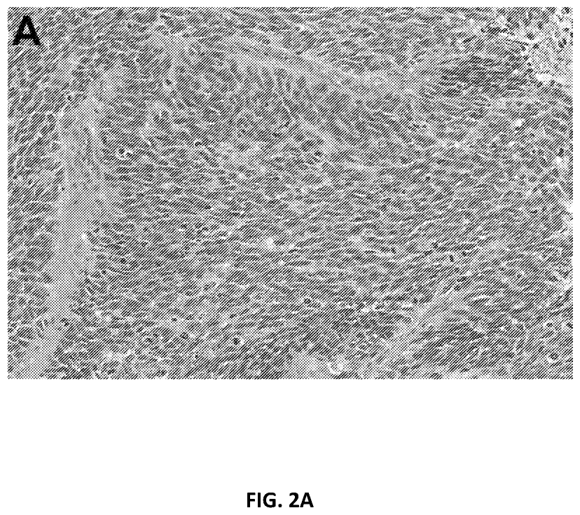

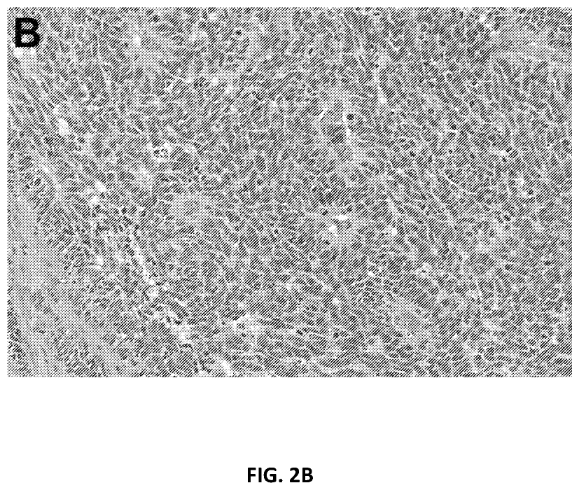

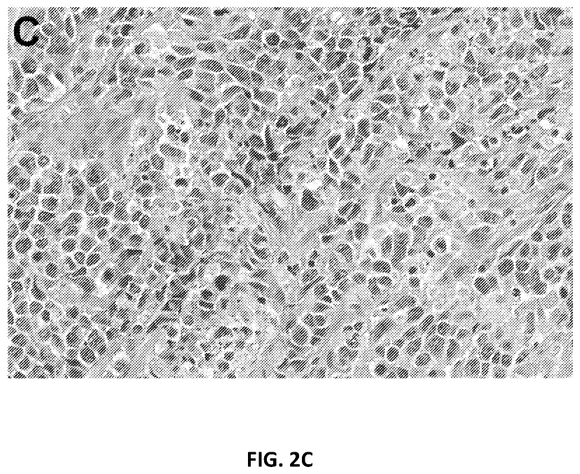

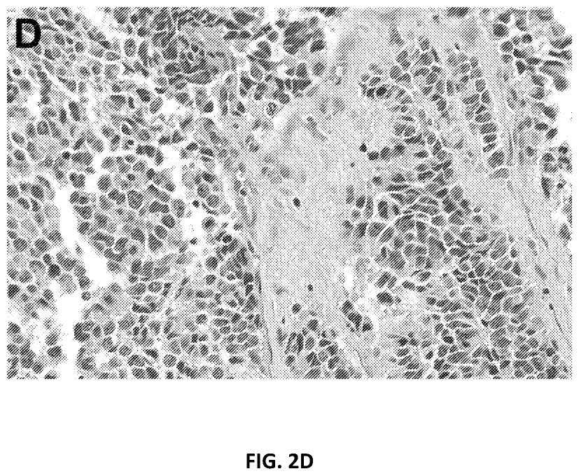

FIGS. 2A-2D show representative images of liver metastases from an ENET case (case number 12) arising from testicular teratoma. The patient was a 31-year-old male with recurrent metastasis to the liver and prior history of testicular ENET arising in teratoma. The tumor exhibited gain of chromosome 12p, as well as a PIK3CA E542K mutation and a BCOR-CLIP2 fusion. FIG. 2A shows that the tumor cells were predominantly uniform, oval to spindle-shaped with scant cytoplasm and delicate chromatin. FIG. 2B shows perivascular pseudorosettes were easily identified. FIG. 2C shows areas with nuclear pleomorphism and moderate pale eosinophilic cytoplasm. FIG. 2D shows focal myxoid or fibrillary formation.

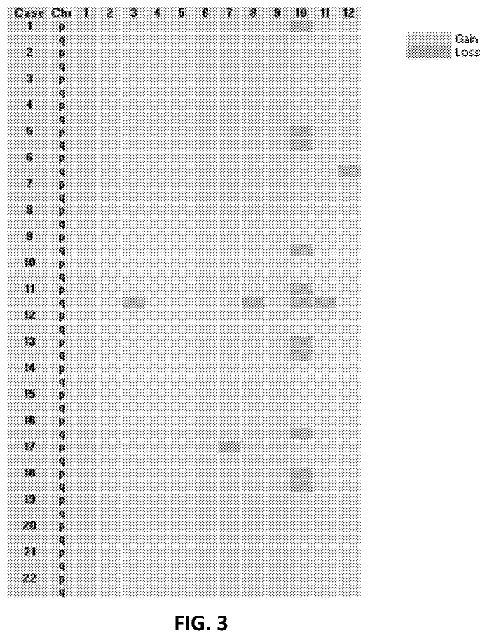

FIG. 3 provides chromosomal copy number changes observed in the 12 patients with advanced testicular ENETs of GCT origin in this study.

### CONCLUSIONS

ENETs arising from GCT are molecularly heterogeneous; however, a subset may be stratified by alterations in 1) MYCN/MYC/TP53/MDM2, 2) PIK3 pathways, or 3) fusions that overlap with molecularly defined CNS-ENETs entities.

BRD4-MAU2 and BCOR-CLIP2 fusions may be indicative of ENETs arising from GCTs in the setting of a testicular tumor. BRD4 and BCOR fusions with other gene partners may also be helpful in diagnosing these tumors.

What is claimed is:

1. A method for assessing or diagnosing a cancer in an individual, comprising detecting a BRD4-MAU2 fusion and/or a BCOR-CLIP2 fusion in a sample from an individual having a cancer, wherein detecting the BRD4-MAU2 fusion and/or the BCOR-CLIP2 fusion in the sample indicates that the cancer is likely an embryonic-type neuroectodermal tumor (ENET).

2. The method of claim 1, wherein the BRD4-MAU2 fusion:
(a) comprises a fusion of a BRD4 gene, or a portion thereof, fused to a MAU2 gene, or a portion thereof;
(b) comprises or results from a BRD4 breakpoint in BRD4 exon 3;
(c) comprises a fusion of BRD4 exon 3, or a portion thereof, fused to an exon of MAU2, or a portion thereof;
(d) results from a breakpoint within chromosomal coordinates chr19:15379580-15379772 and/or chr19:19434185-19434416; and/or
(e) is a BRD4-MAU2 fusion as described in Table 3.

3. The method of claim 1, wherein the BCOR-CLIP2 fusion:
(a) comprises, in the 5′ to 3′ direction, a fusion of a BCOR gene, or a portion thereof, fused to a CLIP2 gene, or a portion thereof;
(b) comprises or results from a BCOR breakpoint in BCOR exon 9 and/or a CLIP2 breakpoint in CLIP2 intron 2;
(c) comprises, in the 5′ to 3′ direction, a fusion of BCOR exon 9, or a portion thereof, fused to CLIP2 exon 3, or a portion thereof;
(d) comprises, in the 5′ to 3′ direction, exons 1-8 and exon 9, or a portion thereof, of BCOR fused to exon 3, or a portion thereof, and exons 4-17 of CLIP2;
(e) results from a breakpoint within chromosomal coordinates chrX:39922132-39922319 and/or chr7:73738770-73738988; and/or
(f) is a BCOR-CLIP2 fusion as described in Table 3.

4. The method of claim 1, wherein detecting the BRD4-MAU2 fusion in the sample identifies the individual or the cancer as likely to respond to a BET bromodomain inhibitor treatment.

5. The method of claim 4, wherein the BET bromodomain inhibitor is JQ1, I-BET762, OTX015, I-BET151, RVX-208, MS417, ABBV-075, ABBV-744, SJ432, AZD5153, INCB054329, or any combination thereof.

6. The method of claim 4, further comprising administering the BET bromodomain inhibitor to the individual based, at least in part, on detecting the BRD4-MAU2 fusion in the sample.

7. The method of claim 1, wherein the BCOR-CLIP2 fusion results in a loss of function of BCOR.

8. The method of claim 1, further comprising administering an anti-cancer therapy to the individual based, at least in part, on detecting the BCOR-CLIP2 fusion in the sample.

9. The method of claim 1, wherein the cancer is a testicular tumor, optionally a testicular tumor arising from a germ cell tumor.

10. The method of claim 1, wherein the cancer is a testicular embryonic-type neuroectodermal tumor arising from a germ cell tumor.

11. The method of claim 1, wherein the cancer is advanced and/or metastatic.

12. A method for treating a cancer, comprising:
(a) detecting a BRD4-MAU2 fusion in a sample from an individual having a cancer, and
(b) administering a BET bromodomain inhibitor to the individual based, at least in part, on detecting the BRD4-MAU2 fusion in the sample.
